(19) United States
(12) Patent Application Publication
(10) Pub. No.: US 2019/0134238 A1
(43) Pub. Date: May 9, 2019

(54) SN-117M LABELED MANNOSE COUPLED DEXTRAN AMINE

(71) Applicant: Serene, LLC, The Woodlands, TX (US)

(72) Inventors: Nigel R. Stevenson, Sugar Hill, GA (US); Jaime Simon, Angleton, TX (US); Lance Cooper, Angleton, TX (US)

(21) Appl. No.: 15/762,118

(22) PCT Filed: Sep. 14, 2016
(86) PCT No.: PCT/US2016/051618

§ 371 Date: Mar. 22, 2018

Related U.S. Application Data
(60) Provisional application No. 62/232,519, filed on Sep. 25, 2015.

(51) Int. Cl. A61K 51/06; A61P 19/02; A61P 35/00; C08B 37/02; A61K 9/00

(52) U.S. Cl. CPC A61K 51/065; A61P 19/02; A61K 9/0019; C08B 37/0021; A61P 35/00

(57) ABSTRACT

Amine modified dextran is labeled with mannose molecules as well as tin-117m. This tin-117m labeled mannose modified dextran is useful in treating maladies that express CD206, in particular rheumatoid arthritis, as well as the cancer typically located in the lymph nodes. This provides a systemic treatment for such maladies. The tin-117m will destroy cells to which it is bonded and also can be imaged. Further, due to the nature of the radiation from the tin-117m, it does not do significant damage to nearby healthy cells.

### RELATED APPLICATIONS

The present application is a national phase application of PCT Application No. PCT/US2016/051618 filed Sep. 14, 2016, which claims priority to U.S. Patent Application No. 62/232,519 filed Sep. 25, 2015, the disclosures of which are hereby incorporated herein by reference in their entirety.

### BACKGROUND OF THE INVENTION

Both rheumatoid arthritis and certain cancers, particularly those located in the lymph nodes express CD206 because they associate with CD206 positive macrophages. There are radiopharmaceuticals designed to bind to CD206 but they only image the arthritis or the cancer cells and provide no therapeutic benefit.

### SUMMARY OF THE INVENTION

The present invention provides a method of imaging, as well as treating, rheumatoid arthritis as well as certain cancers wherein the cancer cells associate with CD206 positive macrophages, or express CD206, in particular, cancer cells typically located in lymph nodes. Applications exist in both humans and animals.

### BRIEF DESCRIPTION OF THE DRAWINGS

The accompanying figures, which are incorporated in and constitute a part of this specification, illustrate exemplary embodiments of the invention and, together with a general description of the invention given above, and the detailed description given below, serve to explain the principles of the invention.

FIG. 1 is a table showing the analysis of mannose conjugation, in accordance with embodiments of the invention.

FIG. 2 is a table showing biodistribution of a Sn-117m composition in mice.

FIG. 3 is a table showing biodistribution of a Sn-117m composition in mice.

FIG. 4 is a table showing biodistribution of a Tc-99m composition in mice.

FIG. 5 is a table showing biodistribution of a Tc-99m composition in mice.

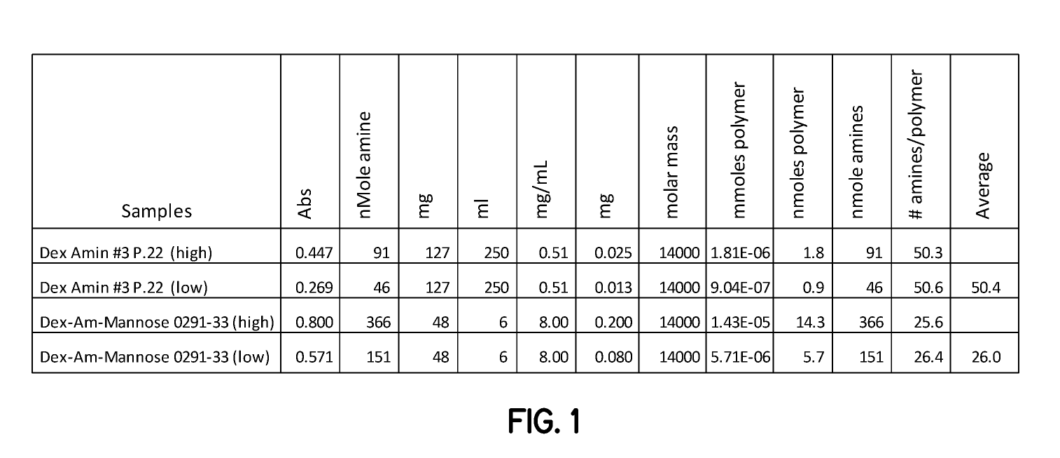

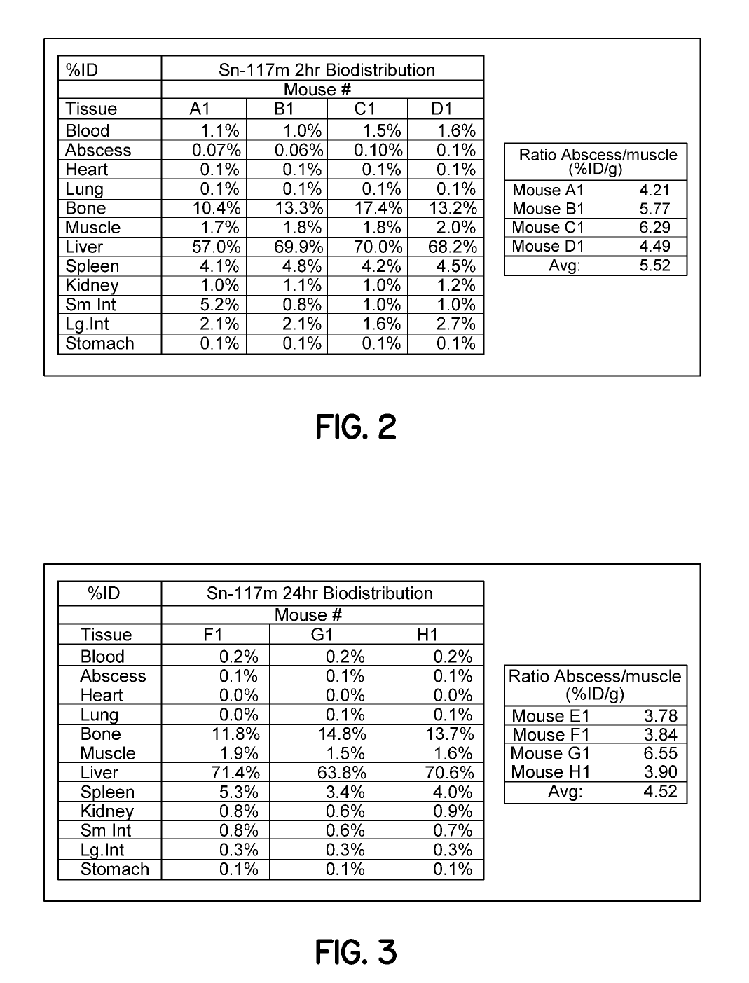

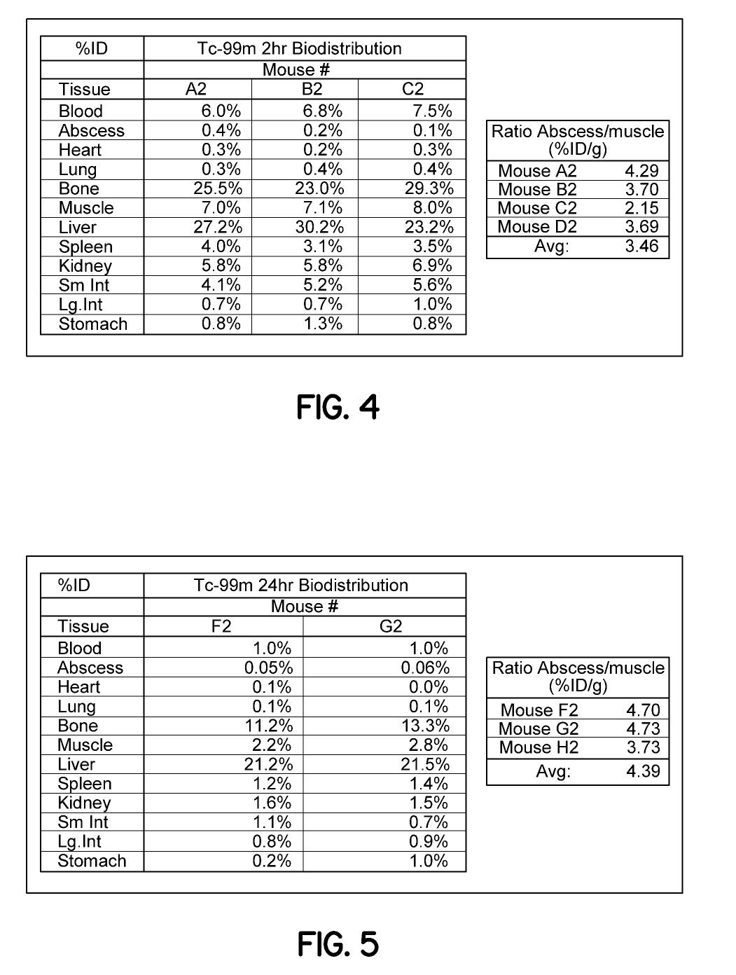

The composition is tin-117m labeled mannose coupled dextran amine. In particular, tin-117m-isothiocyanato-benzyl-DOTA can be bonded to mannose coupled dextran amine. The tin-117m is a gamma emitter as well as a conversion electron emitter. The gamma particle can be imaged. The conversion electron is effective to reduce inflammation caused by rheumatoid arthritis and will deliver a radiation dose to cancer cells within a distance of ˜300 microns that may be lethal to those cells. In low doses (i.e., 10× to 100× lower than the radiation necrosis dose or non-DNA and non-RNA fracturing dose described below) a cellular apoptotic hormesis effect has been noted with these conversion electrons and is applicable to rheumatologic and cancer conditions. The invention will be further appreciated in light of the following detailed description:

### DETAILED DESCRIPTION OF THE INVENTION

The composition of the present invention is an amine modified dextran chain with both mannose and tin-117m bonded to the various amine groups of the dextran. The amine modified dextran is more particularly disclosed in U.S. Pat. No. 6,409,990, the disclosure of which is hereby incorporated by reference. It is also commercially available under the trademark MANOCEPT®. Dextran is a natural product derived from bacteria. It is isolated in high molecular weight form and can be hydrolyzed and purified in controlled fashion to various smaller molecular weights, for example, molecular weight of 1000; 10,000; 40,000; 70,000; 110,000; 150,000 and 500,000. Each of the listed molecular weight species can be used with the present invention and each may have more or less suitable effect for a given application. For tumor imaging and treatment, one would select dextran with a size that, after conjugation of the amine leashes, would have a final molecular weight of 50 to 70 kilodaltons. Other molecular weights suitable for use with rheumatoid arthritis and cancer treatment include 10 kDa; 15 kDa; 20 kDa; 30 kDa; 40 kDa; 50 kDa and higher. One particular range is from 10 kDa to 30 kDa. Commercially available amine modified dextran has a molecular weight of approximately 14 kDa.

The dextran molecule has a large number of available hydroxyl groups. Depending on the size of the dextran, these can number in the hundreds. Amine groups are bonded to the hydroxyl groups of the dextran by activation with, for example, allyl bromide. The allyl groups are subsequently reacted with aminoethanethiol and DMSO to produce amine terminated leashes as further described in U.S. Pat. No. 6,409,990. This compound can then be combined with tin-117m and mannose to form the composition for use in the present invention.

The tin-117m can be carrier or no carrier added tin-117m. Further, it can be high specific activity tin-117m as well as low specific activity tin-117m. High specific activity tin-117m is generally tin-117m with an activity of at least 100 Ci per gram, preferably at least 1000 Ci per gram or 10,000 Ci per gram or 15,000 Ci per gram or 20,000 Ci per gram or higher. As described hereinafter, generally high specific activity no-carrier-added tin-117m is utilized in the present invention, although in certain applications, carrier-added, low specific activity tin-117m can be used.

No-carrier-added tin-117m can be prepared in an accelerator, such as cyclotron, by transmutation of antimony into no-carrier-added tin-117m by high-energy proton induced nuclear reactions. No-carrier-added tin-117m can also be obtained by exposing cadmium 116 to an alpha particle beam as described in U.S. Pat. No. 8,257,681, the disclosure of which is incorporated herein by reference. This permits formation of high specific activity tin-117m, preferably having 100-1000 or more curies per gram. Current methods provide for 20,000 Ci/g.

The tin labeled dextran is formed by first binding mannose molecules to a dextran chain. Next the aminobenzyl-DOTA is combined with high specific activity tin-117m. The tin-117m complexed aminobenzyl-DOTA is reacted with the mannose modified dextran chain to form the imaging/treatment agent of the present invention.

The chemical attachment of mannose to amino dextran can be accomplished by number of different methods, for example, that described for attachment to human serum albumin (Vera et al, (1985) J and UCL-. MED 26:1157-1167) and that described for attachment two Polylysine (Vera et al. (1995) ACAD. RAD IOL. 2:497-596). A specific example of the bonding mannose to the amine labeled dextran is disclosed in the example below. Generally in such a reaction, less than all of the available amine leashes are linked to mannose groups, leaving other unreacted amine leashes available to bond to the tin complexes as well as other compounds.

The formation of the tin-117m complexed aminobenzyl-DOTA is further disclosed in detail in the Journal of Radioanalytical and Nuclear Chemistry: Volume 305, Issue 1 (2015), Page 99-108 (DOI: 10.1007/s10967-015-4031-7) as well as U.S. Pat. No. 8,283,167, the disclosure of which is hereby incorporated by reference. As indicated, the tin can be carrier-added or no-carrier-added, high specific activity or low specific activity, but as disclosed hereinafter the high specific activity no-carrier-added tin-117m as formed by the method described in U.S. Pat. No. 8,257,681 is used in the present invention.

In certain embodiments, the tin mannose bonded dextran is further reacted with a charge containing group which reduces liver uptake. Any charged molecule which is suitable for use in radiopharmaceuticals can be used in the present invention. In particular, acid or ester containing compositions are particularly suitable, such as, for example diethylenetriaminepentaacetic acid (DTPA). The DTPA modified dextran can be formed by first activating the DTPA with isobutyl chloroformate. This is carried in acetonitrile at −30° C. The activated DTPA is slowly added to the amino terminated dextran together with bicarbonate solution at about 4° C. The solution is stirred overnight at room temperature. After extensive diafiltration of the product with five exchange volumes of bicarbonate buffer followed by five exchange volumes of deionized water, the retentate is concentrated and freeze-dried. The charge containing molecule (DTPA) bonds to unreacted amine leashes on the dextran molecule. Alternatively, the di-anhydride of DTPA can be used in a similar manner. In addition, derivatization of some of the amine groups in the polymer with polyethers such as polyethylene glycols could be used to enhance hydrophilicity in order to increase the blood retention of the final construct.

The compound of the present invention is utilized to treat arthritis or cancer which expresses CD206 by injecting the compound of the present invention carried in an appropriate carrier, such as saline, into the mammal. With tin-117m, there are two potential dosage regimens. The first is a dosage intended to disrupt cell DNA causing apoptosis. Generally, such a dosage will be from about 0.05 milli curies to 40 mCi, generally 1 mCi to 10 mCi. This can be injected intravenously, intra-articularly, subcutaneously, intra-lymphatically and intrathecally and can be repeated periodically as needed. The composition can be re-injected at about one month intervals if needed.

Further, the compound of the present invention can be administered at a hormetic dose. The hormetic dose is designed to be low enough to activate the immune response of the mammal to affect apoptosis of the affected cells. Generally, the hormetic dose will be from 1/10 to 1/100 of the normal dose and will generally be from about 0.0005 micro curies to about two mCi or to less than 1 mCi, more typically about 0.005 mCi to about 4 mCi (depending on the mode of administration). The present invention can also be administered for continuous treatment of chronic arthritis. This can be administered intravenously, intra-articularly, subcutaneously, intra-lymphatically and intrathecally. The present invention will be further appreciated in light of the following detailed example.

### Reagents:

All aqueous solutions were prepared utilizing in-house deionized water. Sodium bicarbonate was Sigma Aldrich 99.0-100.5% purity. The sodium carbonate used was Sigma Aldrich 99.5% ACS level reagent. Dextran-amine, molecular weight 14kDa with 31 NH₂ groups per molecule, was obtained from Reliable Pharmaceutical. Cyanomethyl-2,3,4,6-tetra-O-acetyl-1-thio-β-D-mannose (CNM-thiomannose) was obtained from Reliable Pharmaceutical. Methanol used was Fisher Scientific Certified ACS 99.9% purity and was stored over molecular sieves, 4 angstrom, 8-12 mesh from Acros Organics. The sodium methoxide used was pure titrant grade, 0.5 M in methanol from Acros Organics. Glycine was from Sigma Life Science, Reagent Plus 99% grade. Glycine standard solution was prepared from deionized water and frozen in between uses to preserve integrity. The 2,4,6-trinitrobenzene sulfonic acid (TNBSA) 5% w/v in methanol was obtained from Thermo Scientific.

### EXAMPLE 1

### Synthesis

Mannose Conjugation. Dextran-amine was conjugated with mannose utilizing imidate coupling. In preparation for this reaction, 0.0772 g CNM-thiomannose was deacetylated with 0.4 ml sodium methoxide (0.5 M) in 10 ml dry methanol at room temperature (approx. 22° C.) under nitrogen atmosphere for 20 hours. This deacetylation was carried out in a 50 ml round bottom flask on a Buchi Rotavapor R-200 rotating at moderate speed for agitation. Upon completion of the deacetylation, and immediately prior to the imidate coupling step, the methanol was removed via vacuum. The coupling reaction was then immediately initiated by the addition of 0.0480 g of Dextran-amine dissolved in 6 ml of 0.05 M sodium carbonate/sodium bicarbonate buffer. This mixture was allowed to react at room temperature (approx. 22° C.) for 22 hours, again utilizing rotation on the Rotavapor for agitation. Upon completion, 72% of the product was transferred to an Amicon® Ultra-15 Centrifugal Filter unit (10,000 NMWL) and dialyzed with 5-ml exchanges of deionized water at 5000 rpm for 50 minutes. This was repeated two additional times. The concentrate was reconstituted with deionized water to a total weight of 1.9936 g and analyzed for amine density by 2,4,6-trinitrobenzene sulfonic acid (TNBSA) assay using glycine as a standard to determine the extent of mannose coupling. Additionally a sample of the original dextran-amine conjugate was analyzed for amine density for comparative purposes.

Mannose conjugation analytical. In order to determine the level of mannose conjugation, the number of free amine groups on the dextran-amine polymer was measured before and after the reaction. Glycine was used to calibrate the colorimetric method utilizing TNBSA¹. Five different glycine concentration samples and a blank were used yielding calibration curve with a correlation coefficient of R2=0.9957. Analysis of the uncoupled dextran amine and the mannose coupled dextran amine material indicated that on average 48.4% of the amine sites had been conjugated with mannose. Table 1 summarizes this data.

### EXAMPLE 2

### Radiolabeling

High specific activity Sn-117m was chelated with the bifunctional chelating agent aminobenzyl-DOTA. First a solution of Sn-117m (about 1-2 mCi) in 4M HCI was placed in a microwave tube and heated while purging with nitrogen until there was no visible volume in the tube. Chelation was accomplished by adding a 20 molar excess of aminobenzyl DOTA to the Sn. The tube was sealed then heated to 140° C. for 15 minutes. After cooling the Sn-117m-ABD formed was purified by high performance liquid chromatography (HPLC) with a reverse phase column. The product peak was collected and treated and the volume reduced to approximately 0.5 ML. This was treated with 0.2 uL of neat thiophosgene and after one minute was extracted 4 times with dimethyl ether to remove unreacted thiophosgene. The same HPLC method was used to show the conversion of the amine to isothiocyanate. The retention time of the radioactive peak shifted from 4 minutes to 17 minutes.

Mannose coupled dextran amine was combined in a 5:1 molar excess over the Sn-117m chelate and the pH was adjusted from 9 to 9.2. The solution was allowed to stand for 90 minutes at 37° C. Purification was accomplished using a 6,000 molecular weight gravity fed size exclusion column. There was baseline separation of the early eluting product peak and the low molecular weight impurities that were more retained by the column. Yields using this process typically ranged from 30 to 60%.

### EXAMPLE 3

### Biodistribution

Biodistribution Preparation. Eight male BALB/c mice, under Isoflurane anesthesia, were each injected with 20 uL of turpentine into the gastrocnemius muscle of the right hind leg using a ⅓ cc insulin syringe. The mice were kept in group housing with the injected leg marked. After 24 hours, the mice were restrained in an open cylinder and each injected with 20 μL of the Sn-117m Composition prepared above into the lateral tail vein using a ⅓ cc insulin syringe. The mice were individually housed in cages with absorbent paper under a wire mesh bottom.

The mice were split into groups of four. The first group were sacrificed at 2 hours after the injection and the other group 24 hours after the injection. Tissues and samples collected were: blood, heart, lung, left femur, left thigh muscle, liver, spleen, kidneys, small intestine, large intestine, stomach, tail, abscess, remainder of carcass and bladder along with all collected absorbent paper containing accumulated feces and urine. The carcass consists of the remaining musculoskeletal structure, reproductive organs, the skin, head and limbs. The samples were then counted in a Nal crystal for 1 minute.

A second experiment was done with another eight male BALB/c mice. They were placed under Isoflurane anesthesia, and each injected with 20 uL of turpentine into the gastrocnemius muscle of the right hind leg using a ⅓ cc insulin syringe. The mice were kept in group housing with the injected leg marked. After 24 hours, the mice were restrained in an open cylinder and each injected with 20 μL of Tc-99m labeled mannose modified dextran into the lateral tail vein using a ⅓ cc insulin syringe. The mice were individually housed in cages with absorbent paper under a wire mesh bottom.

The mice were split into groups of four. The first group was sacrificed at 2 hours after the injection and the other group 24 hours after the injection. Tissues and samples collected were: blood, heart, lung, left femur, left thigh muscle, liver, spleen, kidneys, small intestine, large intestine, stomach, tail, abscess, remainder of carcass and bladder along with all collected absorbent paper containing accumulated feces and urine. The carcass consists of the remaining musculoskeletal structure, reproductive organs, the skin, head and limbs. The samples were then counted in a Nal crystal for 1 minute.

The biodistribution of Sn-117m labeled mannose modified dextran was compared to that of Tc-99m labeled mannose modified dextran. In both cases the abscess to normal muscle ratio was about the same showing that both constructs have similar biological activity. The Sn labeled material had significantly more uptake in the liver than the Tc construct. The Sn labeled material had fewer chelating agents attached and all the chelators had Sn(IV) making them neutrally charged. For the Tc product, there is probably an excess of DTPA over Tc resulting in significantly more chelator (DTPA) with 4 carboxylic acid groups which add to the negative charge of the molecule. Therefore the Sn construct has less charge on the molecule which is the probable reason that it is eliminated from the blood by the liver. To control the rate that the liver clears, the product can be modified by simply adding charged molecules to the polymer such as but not limited to DTPA. Adding DTPA functionality (or another charged group) to the polymer should modify the biological properties to more closely mimic the Tc construct. Alternatively, derivatization of some of the amine groups in the polymer with polyethers such as polyethylene glycols could be used to enhance hydrophilicity in order to increase the blood retention of the final construct.

Based on the above, it is clear that the tin-117m labeled mannose modified dextran will locate cells that express the CD206 protein. Accordingly, once attached to such cells, the tin-117m can be used to image the arthritis or cancer (or both) but also will act to reduce inflammation and, in effect, treat the arthritis and/or cancer. The tin-117m emits a conversion electron which travels approximately 300 μm. So, unlike other radioactive compounds such as strong alpha emitters, it only destroys nearby cells and has no effect on nearby healthy cells. A very low dose of the tin- 117m compound can be administered. This is a hormetic dose which is effective in treating the malady, but presumably by encouraging the body's own immune system to attack the arthritic or cancerous cells. A higher dosage will directly destroy the cells. But since the tin-117m has a fourteen day half-life, after a period of a few weeks, a higher dose of the tin-117m decays to a hormetic dose. Thus, a single treatment can act to treat the malady in both fashions, directly destroying the cells and initiating hormesis.

This has been a description of the present invention, along with the preferred method of practicing the present invention. However, the invention itself should be defined only by the appended claims wherein we claim:

What is claimed is:

1. A method of treating or imaging a malady which expresses CD206 by injecting into a mammal with said malady, a dosage of tin-117m labeled mannose modified dextran, said dosage effective to treat or image said malady.

2. The method claimed in claim 1 wherein said tin-117m is no-carrier-added high specific activity tin-117m.

3. The method claimed in claim 2 wherein said malady is rheumatoid arthritis.

4. The method claimed in claim 1 wherein said malady is cancer.

5. The method claimed in claim 1 wherein said effective dosage is from 0.05 mCi to 40 mCi.

6. The method claimed in claim 1 wherein said effective dosage is a hormetic dosage.

7. A method inducing cellular apoptosis in rheumatologic conditions by administering to a mammal a hormetic dosage of no-carrier-added high specific activity tin-117m labeled mannose modified dextran.

8. A method of inducing cellular apoptosis in cancer cells in a mammal by administering to said mammal a hormetic dosage of no-carrier-added high specific activity tin-117m labeled mannose modified dextran.

9. A compound comprising:
a dextran chain having a plurality of mannose molecules bound to said chain and further having a plurality of tin-117m atoms bound to said dextran chain.

10. The compound of claim 9 wherein said dextran has a molecular weight of 10 to 30 kDa.

11. The compound claimed in claim 9 wherein a plurality of charged molecules are bonded to said dextran chain.

12. The compound claimed in claim 9 wherein said plurality of mannose molecules are attached to said dextran chain by amine leashes.

13. The compound claimed in claim 9 wherein said tin-117m is bonded to aminobenzyl-DOTA which is attached to said dextran chain by amine leashes.

14. The compound claimed in claim 13 wherein said tin-117m is no-carrier-added, high specific activity tin-117m.

15. The compound claimed in claim 9 further comprising hydrophilic groups attached to said dextran chain.

16. The compound claimed in claim 15 wherein said hydrophilic groups are DPTA or the dianhydride of DTPA.
